(19) United States
(12) Patent Application Publication
(10) Pub. No.: US 2023/0337930 A1
(43) Pub. Date: Oct. 26, 2023

(54) ELECTRICAL IMPEDANCE TOMOGRAPHY BASED MEDICAL SCREENING SYSTEM

(71) Applicant: Gense Technologies Limited, Wong Chuk Hang (HK)

(72) Inventors: Russell Wade Chan, Kowloon (HK); Chung San Wong, Tseung Kwan O (HK)

(21) Appl. No.: 18/340,320

(22) Filed: Jun. 23, 2023

Related U.S. Application Data
(63) Continuation-in-part of application No. 16/976,542, filed on Aug. 28, 2020, now Pat. No. 11,705,748.

(30) Foreign Application Priority Data
Feb. 28, 2018 (HK) 18102884.5

(51) Int. Cl. A61B 5/0536; A61B 5/00

(52) U.S. Cl. CPC A61B 5/0536; A61B 5/0013; A61B 5/725; A61B 5/6831; A61B 2560/0431

(57) ABSTRACT

A portable device of a medical screening system comprises a current generation module, a signal distribution and readout module, a data acquisition module, and a control and output module. The current generation module generates electric current signals for providing to a subject. The signal distribution and readout module provides the generated electric current signals to the subject and receives responsive electric signals from the subject, both via a wearable device with electrodes. The data acquisition module processes the responsive electric potential signals received from the subject to determine potential difference signals. The control and output module processes the potential difference signals and transmit the processed signals to a server for determining electrical impedance tomography data and medical screening result.

### CROSS-REFERENCE TO RELATED APPLICATION

This application is a continuation-in-part of U.S. patent application Ser. No. 16/976,542, filed Feb. 27, 2019.

### TECHNICAL FIELD

The invention relates to an electrical impedance tomography based medical screening system.

### BACKGROUND

With the rapid advancement in technologies driven by consumers' craving for increasingly portable devices with advanced functions, wearable devices have become extremely popular in the consumer market in recent years.

### SUMMARY OF THE INVENTION

In a first aspect, there is provided a portable device of a medical screening system, comprising: a current generation module arranged to generate electric current signals for providing to a subject; a signal distribution and readout module arranged to receive and provide the generated electric current signals to the subject via a wearable device with electrodes, and receive responsive electric signals from the subject via the wearable device with electrodes; a data acquisition module arranged to process the responsive electric potential signals received from the subject to determine potential difference signals; and a control and output module arranged to process the potential difference signals and transmit the processed signals to a server for determining electrical impedance tomography data and medical screening result.

In some embodiments, the portable device further comprises: an isolation protection module electrically connected with the current generation module, the signal distribution and readout module, the data acquisition module, and the control and output module.

In some embodiments, the isolation protection module comprises an isolation bridge and a power isolation circuit operably coupled with the isolation bridge.

In some embodiments, the current generation module comprises a waveform generator and a current generator operably connected with the waveform generator.

In some embodiments, the current generation module further comprises a filter operably coupled between the waveform generator and the current generator. The filter is arranged to reduce harmonic distortion and/or electromagnetic interference of wave signals generated by the waveform generator.

In some embodiments, the waveform generator comprises a sinusoidal waveform generator.

In some embodiments, the signal distribution and readout module comprises a plurality of N:1 multiplexers, wherein N is an integer. For example, N is 16 or 32.

In some embodiments, at least one of the plurality of N:1 multiplexers is for signal distribution and at least one of the plurality of N:1 multiplexers is for readout.

In some embodiments, the signal distribution and readout module is arranged to operate based on an adjacent pattern measurement protocol.

In some embodiments, the data acquisition module comprises a data acquisition amplifier and a filter.

In some embodiments, the data acquisition amplifier comprises a multi-stage data acquisition amplifier.

In some embodiments, the filter comprises a bandpass filter.

In some embodiments, the control and output module is further arranged to control operation of the current generation module and the signal distribution and readout module.

In some embodiments, the control and output module comprises: an analog-to-digital converter for digitizing the potential difference signals received from the data acquisition module; a controller arranged to process the digitized signals; and a communication device operably connected with the controller for communicating the processed signals to the server.

In some embodiments, the controller is further arranged to control operation of the current generation module and the signal distribution and readout module.

In some embodiments, the portable device further comprises: a power management module arranged to manage power provided to the current generation module, the signal distribution and readout module, the data acquisition module, and the control and output module.

In some embodiments, the power management module comprises a power circuit arranged to be electrically connected with a power source.

In a second aspect, there is provided a portable system of a medical screening system, comprising: the portable device of the first aspect, and a wearable device with electrodes arranged to be worn on a body of a user, the wearable device being electrically connectable to the portable device. In some embodiments, the electrodes are removably connected with the wearable device.

In a third aspect, there is provided medical screening system comprising: the portable system of the second aspect, and one or more processors for processing processed signals received from the portable system for determining electrical impedance tomography data and medical screening result. The one or more processor may be provided by a server, such as a cloud computing server.

Other features and aspects of the invention will become apparent by consideration of the detailed description and accompanying drawings. Any feature(s) described herein in relation to one aspect or embodiment may be combined with any other feature(s) described herein in relation to any other aspect or embodiment as appropriate and applicable.

Terms of degree such that “generally”, “about”, “substantially”, or the like, are used, depending on context, to account for manufacture tolerance, degradation, trend, tendency, imperfect practical condition(s), etc. For example, when a value is modified by terms of degree, such as “about”, such expression may include the stated value ±15%, ±10%, ±5%, ±2%, or ±1%.

### BRIEF DESCRIPTION OF THE DRAWINGS

Embodiments of the invention will now be described, by way of example, with reference to the accompanying drawings in which:

FIG. 1 is a flow diagram showing a gesture recognition method implemented using a wearable gesture recognition device in accordance with one embodiment of the invention;

FIG. 2 is an illustration of a wearable gesture recognition device, in the form of a wristband, in accordance with one embodiment of the invention;

FIG. 3A is a front view of a wearable gesture recognition device, in the form of a watch, in accordance with one embodiment of the invention;

FIG. 3B is a rear view of the wearable gesture recognition device of FIG. 3A

FIG. 4 is a functional block diagram of a wearable gesture recognition device in accordance with one embodiment of the invention;

FIG. 5 is a functional block diagram of a server, in the form of a cloud computing server, in accordance with one embodiment of the invention;

FIG. 6 is an illustration of a charger for the wearable gesture recognition device of FIG. 2 in accordance with one embodiment of the invention;

FIG. 7 is an illustration of a ring accessory arranged to be used with the wearable gesture recognition device of FIGS. 2 to 3B in accordance with one embodiment of the invention;

FIG. 8A is a system incorporating a wearable gesture recognition device in accordance with one embodiment of the invention;

FIG. 8B is a system incorporating a wearable gesture recognition device in accordance with one embodiment of the invention;

FIG. 8C is a system incorporating a wearable gesture recognition device in accordance with one embodiment of the invention;

FIG. 8D is a system incorporating a wearable gesture recognition device in accordance with one embodiment of the invention;

FIG. 9 is a schematic diagram of a medical screening system in some embodiments of the invention;

FIG. 10 is another schematic diagram of the medical screening system of FIG. 9;

FIG. 11 is a block diagram of a wearable device for facilitating performing of electrical impedance tomography in some embodiments of the invention;

FIG. 12 is a schematic diagram of a wearable device for facilitating performing of electrical impedance tomography in some embodiments of the invention;

FIG. 13 is a schematic diagram (perspective view) of a wearable belt for facilitating performing of electrical impedance tomography in one embodiment of the invention;

FIG. 14 is a block diagram of a portable system for facilitating performing of electrical impedance tomography in some embodiments of the invention;

FIG. 15A is a block diagram of a portable device of the portable system of FIG. 14 in some embodiments of the invention;

FIG. 15B is a schematic diagram of an adjacent pattern measurement protocol in some embodiments of the invention;

FIG. 16 is a block diagram of a portable device for facilitating performing of electrical impedance tomography in some embodiments of the invention;

FIG. 17 is a block diagram of a portable device for facilitating performing of electrical impedance tomography in some embodiments of the invention;

FIG. 18 is a block diagram of an information handling system in some embodiments of the invention;

FIG. 19 is a schematic diagram illustrating operation of a medical screening system in some embodiments of the invention;

FIG. 20A is a schematic diagram illustrating an example use of a portable system for facilitating performing of electrical impedance tomography;

FIG. 20B is a schematic diagram illustrating an example use of a portable system for facilitating performing of electrical impedance tomography; and

FIG. 20C is a schematic diagram illustrating an example use of a portable system for facilitating performing of electrical impedance tomography.

### DETAILED DESCRIPTION

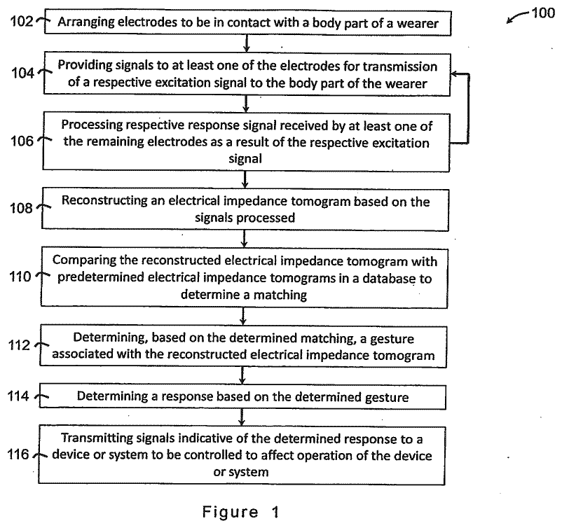

FIG. 1 shows a gesture recognition method 100 implemented using a wearable gesture recognition device in accordance with one embodiment of the invention. The method begins in step 102, wherein a wearable gesture recognition device is worn by a user. The wearable gesture recognition device includes electrodes arranged to be arranged on a body part of the user. In a preferred embodiment, the body part may be a wrist. The electrodes may be contact type or non-contact type.

In step 104, signals are provided to at least one of the electrodes for transmission of a respective excitation signal to the body part of the wearer. The excitation signal may attenuate as it travels through the body part of the wearer. The signals provided may comprise 30 kHz to 50 kHz waveform. The excitation signal may have a combination of different frequency, phase, amplitude, etc. For example, the excitation signal may be formed by waveforms of (1) different shape: square wave, rectangular wave, triangular wave, comb wave, sinusoidal wave, etc.; different sweeping frequency or amplitude: chirp function, etc.; (2) different modulation: amplitude modulation or frequency modulation; or (4) any of their combination. In one example, one of the electrodes is arranged to transmit an excitation signal to the body part of the wearer. In another example, two electrodes are arranged to simultaneously transmit respective excitation signals to the body part of the wearer. The two signals may have same or different properties. The excitation signal may attenuate as it travels through the user.

In step 106, one or more of the remaining electrodes not used for transmission may receive response signal as a result of the respective excitation signal. In one example, the excitation may travel through the body part of the user and picked up by one or more of the remaining electrodes. The time that the response signal is received may be different for different electrodes.

Preferably, steps 104 and 106 are repeated with different electrodes acting as transmission electrode and receiving electrodes, to obtain more information on the response provided by the body part of the user. In one example, the transmission and receive may even be repeated for the same electrodes.

After obtaining sufficient data or information in steps 104 and 106, or after completing an excitation cycle in steps 104 and 106, the method proceeds to step 108, in which an electrical impedance tomogram is reconstructed based on the signals received and processed. The reconstruction may be performed at the wearable gesture recognition device or may be performed at a server or external electronic device operably connected with the wearable gesture recognition device.

Then, in step 110, the reconstructed electrical impedance tomogram is compared with predetermined electrical impedance tomograms in a database to determine a matching. More particular, the reconstructed electrical impedance tomogram is compared with predetermined electrical impedance tomograms in a database to determine which predetermined electrical impedance tomogram is most similar to the reconstructed electrical impedance tomogram. The database may be provided the wearable gesture recognition device, or the server or external electronic device, or both. In one embodiment, the database may be trained based on machine learning method using the processed signals and the reconstructed electrical impedance tomogram. With training, the database can be trained to improve the comparison speed and accuracy.

In step 112, based on the determined matching, the gesture associated with the reconstructed electrical impedance tomogram is determined. The determination may be based on a look-up from the database or a separate database, which associates different predetermined electrical impedance tomogram with predetermined gestures.

In step 114, based on the gesture determined, a response is determined. For example, the response may be determined by looking up a database that associates different predetermined gesture with predetermined responses. The response in the present embodiment may be a control signal to affect operation of an external electronic device or system, or may be a signal to generate a result on an external electronic device or system. In step 116, signals indicative of the determined response is transmitted to a device or system to be controlled to affect operation of the device or system.

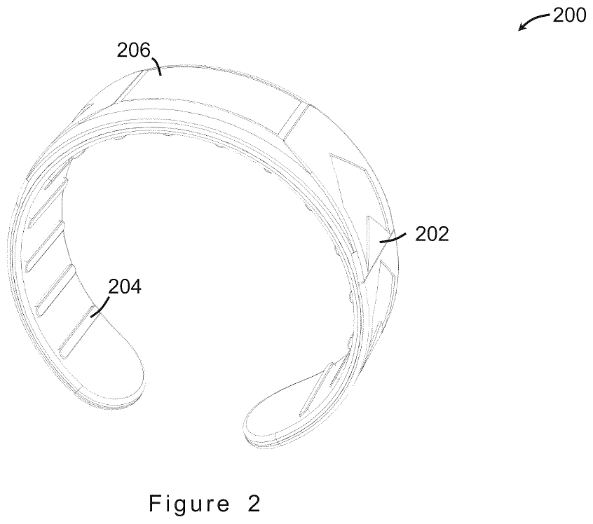

FIG. 2 shows a wearable gesture recognition device, in the form of a wristband 200, in accordance with one embodiment of the invention. The wristband 200 includes a flexible body 202 arranged to be worn by the wearer. Preferably, the flexible body 202 is arranged to be fit onto the wearer by inherent resilience. The flexible body 202 may thus be adapted to be worn by users with different wrist sizes.

Multiple electrodes 204, operable as both transmission and receiving electrodes, are arranged on the inner surface of the wristband 200. The electrodes 204 are in the form of strips that are spaced apart from each other. In the present embodiment, the electrodes 204 are spaced apart substantially equally. However, in some embodiments this is not necessary. The number of electrodes 204 may any number larger than 2. The electrodes 204 may be made of copper, aluminum, or metal alloy. A display 206 is arranged on the outer surface of the wristband 200. The display 206 may be touch sensitive to provide a means for the user to interact with (provide input to) the wristband 200. Various internal structure of the wristband 200 will be described in further detail below.

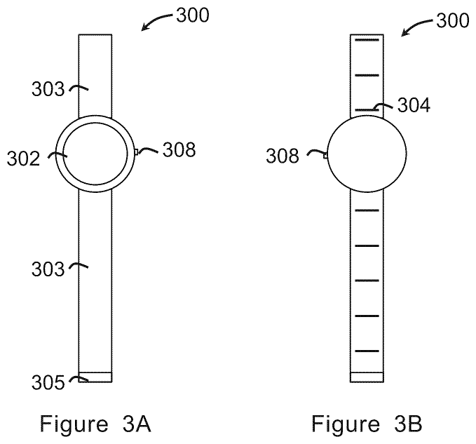

FIGS. 3A and 3B show a wearable gesture recognition device, in the form of a watch 300, in accordance with one embodiment of the invention. The watch 300 includes a watch face 302 providing a display. Flexible watch straps 303 are connected to the watch face. Preferably, a clasp or connector 305 is provided at the open ends of at least one of the watch strap to allow the watch to be worn. Multiple electrodes 304, operable as both transmission and receiving electrodes, are arranged on the inner surface of the watch straps 303. The watch 300 also includes an actuator 308 for receiving user input. The actuator 308 may be in the form of a dial, a button, a slider, etc. Various internal structure of the watch 300 will be described in further detail below.

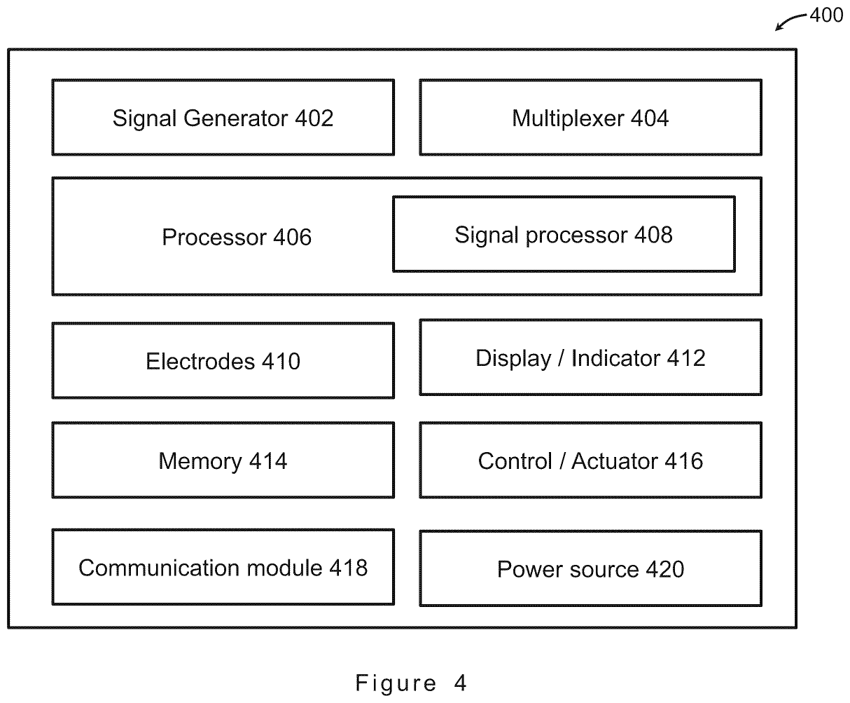

FIG. 4 shows functional block diagram of a wearable gesture recognition device 400 in accordance with one embodiment of the invention. The wristband 200 and watch 300 in FIGS. 2-3B may include like or the same configuration as that illustrated in FIG. 4.

The device 400 includes electrodes 410 arranged to be arranged on a body part of a wearer. The electrodes 410 may be the same as the electrodes 204, 304 shown in FIGS. 2-3B. The electrodes 410 may each be adapted to operate as both transmission electrode and receiving electrode. A multiplexer 404 is arranged to select at least one of the electrodes 410 as transmission electrode and to select at least one of the remaining electrodes as receiving electrode. The multiplexer may be controlled by the processor 406, to implement a predetermined electrode excitation scheme, to select different electrodes 410 as transmission electrode at different instances.

A signal generator 402, e.g., in the form of a waveform generator, is arranged to provide a waveform signal to the electrode(s) selected to be transmission electrode for transmission of a respective excitation signal to the body part of the wearer. In operation, the signal generator 402 may provide different waveform signals to different transmission electrode, and it may transmit waveform signals to multiple transmission electrodes at the same time. Upon transmission of the excitation signals, one or more of the remaining electrodes 410 may be selected as receiving electrodes to receive response signal as a result of the respective excitation signal.

A signal processor 408, as part of a processor 406, is arranged to process the respective response signal received by at least one of the remaining electrodes as a result of the respective excitation signal, for determination of an electrical impedance tomogram for gesture recognition. The signal processor may perform various signal processing, comprising ADC, DAC, noise suppression, SNR boost, filtering, etc. The data processed by the signal processor may either be transmitted to an external electronic device or server through the communication module for further processing, or may be further processed by the processor 406. The further processing comprises determination of the electrical impedance tomogram for gesture recognition, preferably using one or more of the method steps 108-116 in FIG. 1.

In the present embodiment, the processor may be implemented using one or more MCU, controller, CPU, logic gates components, ICs, etc. In one embodiment, the processor is further arranged to process signals and data associated with the determined gesture, the determined response associated with the determined gesture, etc.

The device 400 also includes a memory module 414. The memory module 414 may include a volatile memory unit (such as RAM), a non-volatile unit (such as ROM, EPROM, EEPROM and flash memory) or both. The memory module 414 may be used to store program codes and instructions for operating the device. Preferably, the memory module 414 may also store data processed by the signal processor 408 or the processor 406.

A display or indicator 412 may be provided in the device 400. The display 412 may be an OLED display, a LED display, a LCD display. The display 412 may be touch-sensitive to receive user input. In some embodiments, the device 400 may include indicators in the form of, e.g., LEDs.

The device 400 may also include one or more actuators 416 arranged to receive input from the user. The actuators 416 may be any form and number of buttons, toggle switch, slide switch, press-switch, dials, etc. The user may turn on or off the device 400 using the actuators 416. The user may input data to the device 400 using the actuators 416.

A power source 420 may be arranged in the device 400 for powering the various modules. The power source may include Lithium-based battery. The power source 420 is preferably a rechargeable power source. In one example, the rechargeable power source may be recharged through wired means such as charging port provided on the device. Alternatively, the rechargeable power source may be recharged wirelessly through induction.

The device 400 includes a communication module 418 arranged to communicate information and data between the wearable gesture recognition device 400 and one or both of: an external electronic device and a server. The external electronic device may be a mobile phone, a computer, or a tablet. The server may be a cloud computing server that is preferably implemented by combination of software and hardware. The communication module may be a wired communicate module, a wireless communication module, or both. In the embodiment with a wireless communication module, the module 418 preferably includes a Bluetooth module, in particular a Low energy Bluetooth module. However, in other embodiments, the wireless communication module may alternatively or also include LTE-, Wi-Fi-, NFC-, ZigBee-communication modules.

In one embodiment of the invention, the communication module 418 is arranged to transmit, to the external electronic device or the server, signals processed by the signal processor, for determination of the electrical impedance tomogram for gesture recognition. The communication module 418 may also be arranged to receive, from the external electronic device or the server: signals indicative of a gesture determined based on the determined electrical impedance tomogram, or signals indicative of a response determined based on the determined electrical impedance tomogram.

A person skilled in the art would appreciate that the modules illustrated in FIG. 4 can be implemented using different hardware, software, or a combination of both. Also, the device may include additional modules or include fewer modules (some omitted).

Although not clearly illustrated, the various modules in the device 400 are operably connected with each other, directly or indirectly.

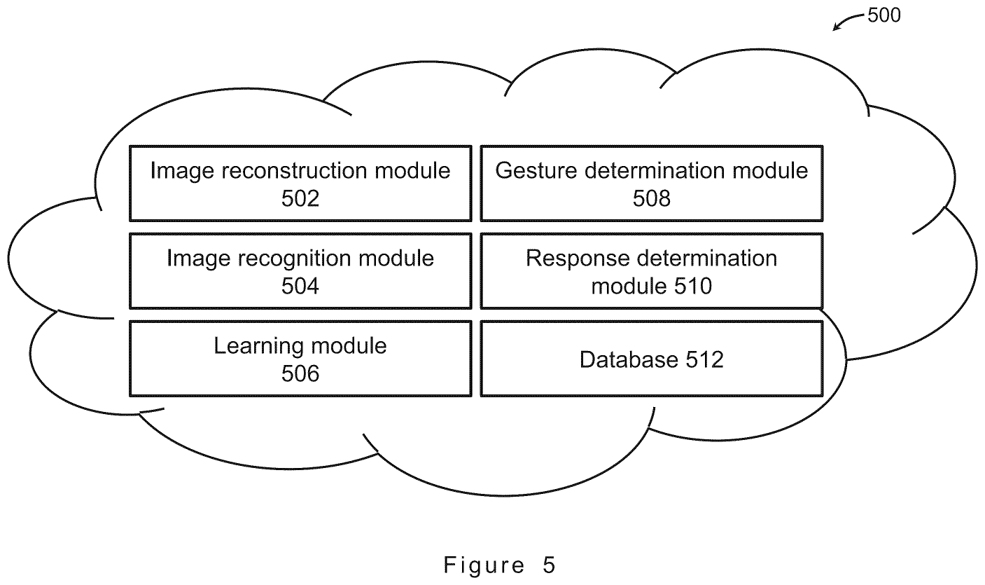

FIG. 5 shows a server 500, in the form of a cloud computing server, in accordance with one embodiment of the invention, arranged to operate with the devices 200, 300, and 400 of FIGS. 2-4.

The server 500 is arranged to communicate data with the device 200, 300, 400, directly, or indirectly through an external electronic device. In one embodiment, the server 500 is arranged to receive, from the device 200, 300, 400, signals processed by the signal processor 408, for determination of the electrical impedance tomogram for gesture recognition. In another embodiment, the server 500 is arranged to receive, from the external electronic device operably connected with the device 200, 300, 400, signals processed by the signal processor 408, for determination of the electrical impedance tomogram for gesture recognition

The server 500 includes an image reconstruction module 502 arranged to reconstruct an electrical impedance tomogram based on signals received from the communication module of the device 200, 300, 400. The reconstruction may include performing back-projection, SNR boost, artifact correction, image correction, registration, co-registration, normalization, etc.

The server 500 also includes an image recognition module 504 arranged to compare the reconstructed electrical impedance tomogram with predetermined electrical impedance tomograms in a database 512 to determine a matching. The predetermined electrical impedance tomograms in the database each correspond to a respective gesture. The image recognition module 504 determines the predetermined electrical impedance tomogram that is most similar to the reconstructed electrical impedance tomogram. In one example, the image recognition module 504 may determine that there is no matching, in which case a response maybe provided back to the device 200, 300, 400, or the external electronic device operably connected with the device 200, 300, 400.

The gesture determination module 508 determines, based on the determined matching result provided by the image recognition module, a predetermined gesture associated with the reconstructed electrical impedance tomogram. The predetermined gesture and its associated with the predetermined electrical impedance tomogram may be set by the user, using an application on an external electronic device, and stored in the server.

The server also includes a response determination module 510 arranged to determine a response based on the determined gesture. The response associated with respective gesture is predetermined, e.g., set by the user, using an application on an external electronic device, and stored in the server. The response determination module 510 may transmit signals indicative of the determined response to a device or system to be controlled to affect operation thereof. Alternatively, the response determination module 510 may transmit signals indicative of the determined response to the device 200, 300, 400, which in turn provides control signal to the device or system to be controlled to affect operation thereof.

Preferably, the server 500 includes a training module 506 that learns, using machine learning method, based on signals received from the communication module, the reconstructed electrical impedance tomogram, the matching result, etc. The training module 506 trains the database 512 accordingly to improve matching accuracy and speed.

A person skilled in the art would appreciate that one or more of the modules in the server 500 may be implemented on the device 200, 300, 400, on an external electronic device connected to the device 200, 300, 400, or on both.

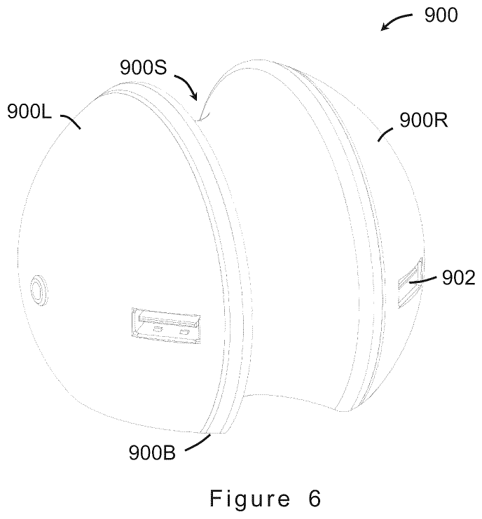

FIG. 6 shows a charger 900 for the device 200 in one embodiment of the invention. The charger 900 has a body with flat base 900B and two generally hemi-spherically shaped sides 900L, 900R. An annular slot 900S is arranged between the two hemi-spherically shaped sides 900L, 900R for receiving the device 200. Means for securing the device 200 to the charger slot 900S may include a mechanical lock, a magnetic lock, etc. In one example, the device 200 includes a magnetic lock member and the charger includes, in the slot 900S, corresponding magnetic lock member that can lock and align the device 200 in the slot 900S. On two sides of the body are USB ports, for receiving data/power from an external electronic device, or for transmitting data/power to an external electronic device, through a cable. In other embodiments the USB ports may be replaced with data/power ports of other standards, e.g., lightning port. The charger 900 may incorporate or be an information handling system described in further detail below.

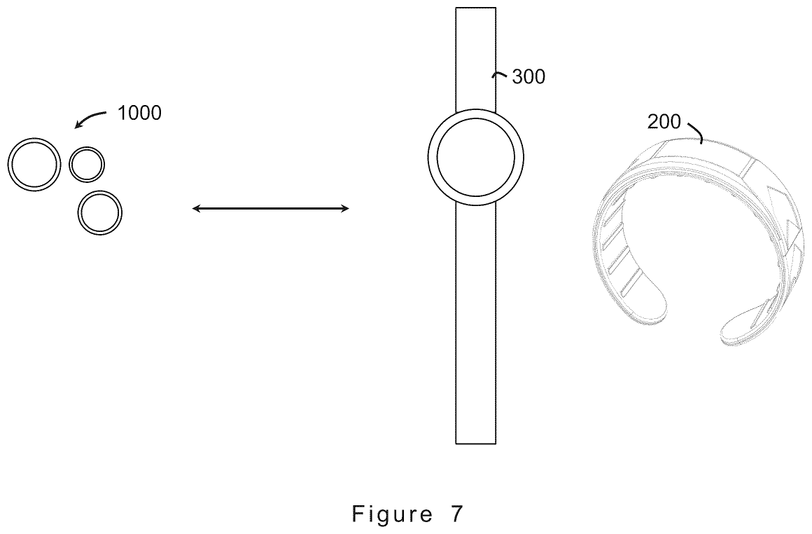

FIG. 7 shows a ring 1000 arranged to operate with the device 200, 300 to improve the measurement accuracy or functions of the device 200, 300. The ring 1000 may be suitably sized to eh worn on a finger of the user. In one embodiment, the ring 1000 may be of like construction of the device 200, 300. The ring 1000 may be arranged to communicate with the device 200, 300 using Bluetooth, near field communication, or other wireless communication protocol. The ring may include electrodes, which function as a reference point, or as those on the device 200, 300, to provide improved gesture recognition accuracy. In some embodiments, the ring 1000 may incorporate or be an information handling system described in further detail below.

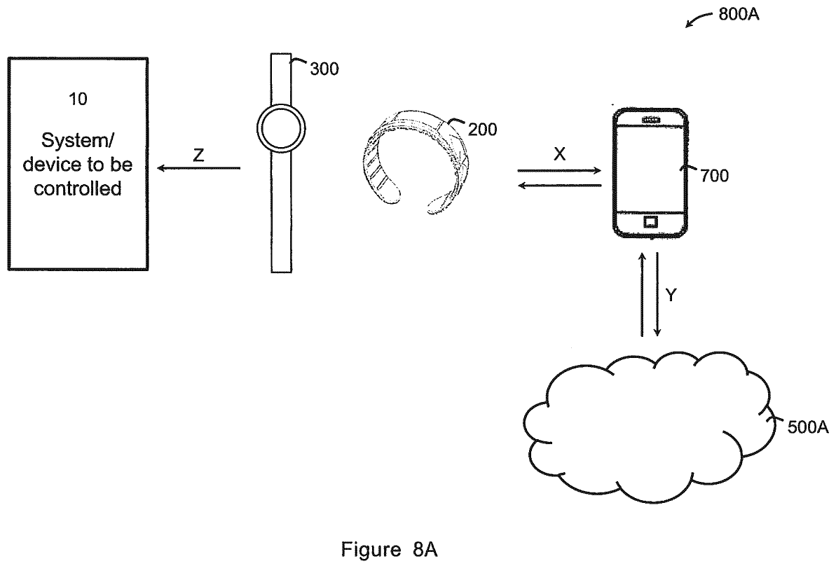

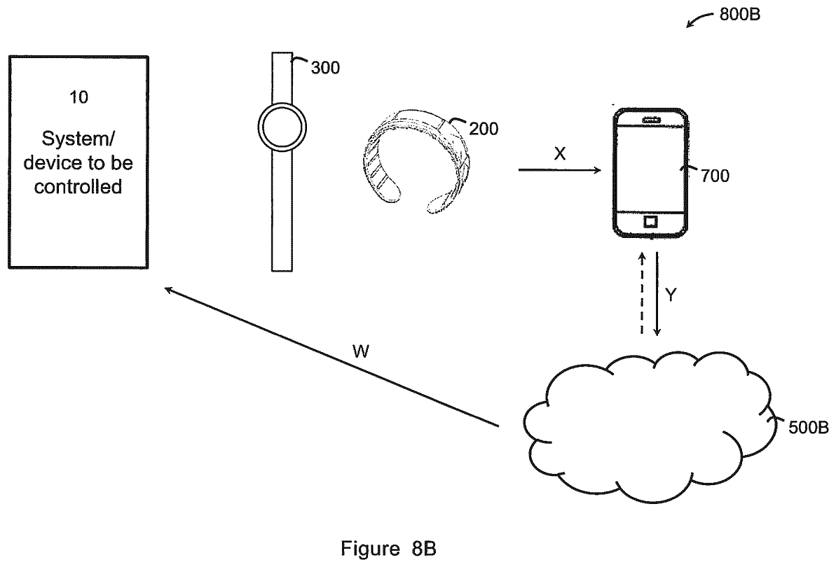

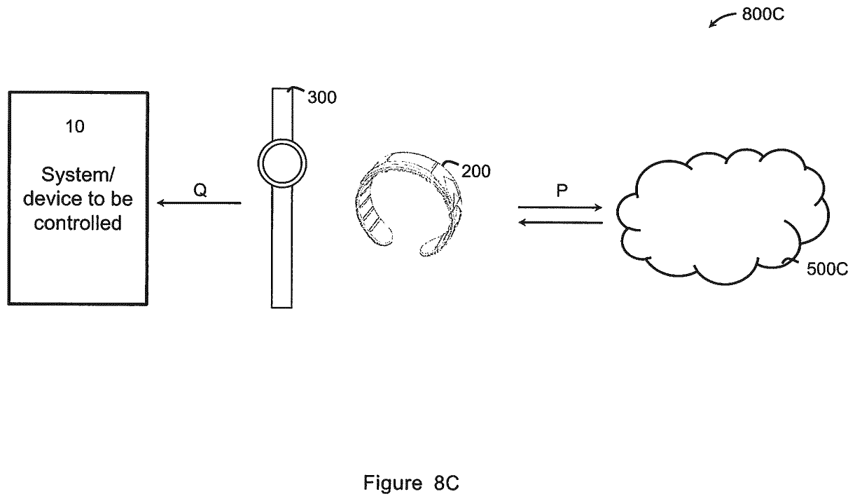

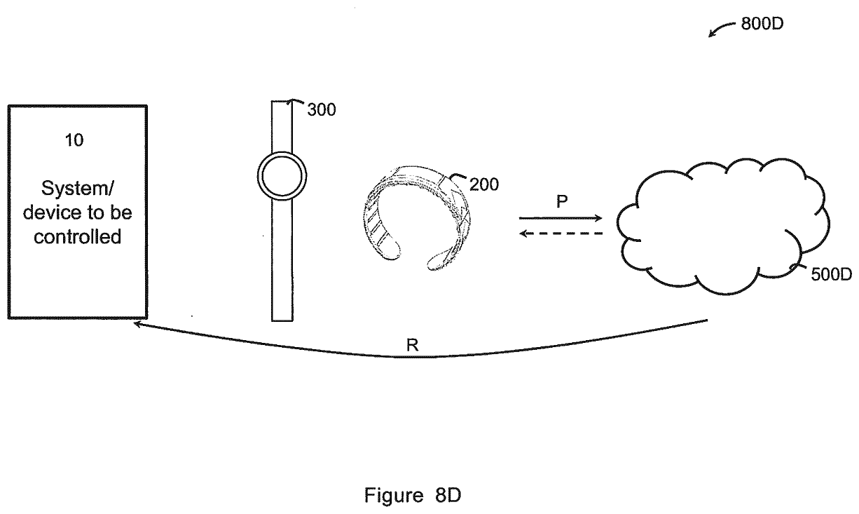

FIGS. 8A to 8D illustrated various systems incorporating a wearable gesture recognition device 200, 300 in accordance with one embodiment of the invention. Systems 800A-800B include the wearable gesture recognition device 200, 300, an external electronic device 700 in the form of a mobile phone, a server 500A-500D with similar or the same construction of server 500, and a system or device to be controlled based on the recognized gesture 10. Systems 800C-800D include all these components except the external electronic device 700. In these embodiments, the system or device to be controlled based on the recognized gesture 10 may be any computing system, e.g., smart phone control module, smart home control module, computer, etc.

The embodiment of the system 800A in FIG. 8A, the device 200, 300 in system 800A detects response signal received in response to the excitation signals provided by the electrodes. The device 200, 300 transmits the processed signal to the smart phone 700 and hence to the server 500A. The communication link X between the device 200, 300 and the phone 700 may be a wireless communication link such as a Bluetooth communication link. The communication link Y between the phone 700 and the server 500A may be a wireless communication link such as a cellular communication link. The server 500A in this example may be arranged to process the processed signal transmitted from the device 200, 300, for: reconstruction of an electrical impedance tomogram, determination of gesture associated with the reconstructed electrical impedance tomogram, determination of response based on the determined gesture, etc. The server 500A may perform one or more of these steps and transmit the result to the device 200, 300 or the phone 700, via links X and Y, for performing the remaining steps. In this embodiment, the server 500A transmits signals indicative of the determined response to the device 200, 300, which in turn provide a control signal via communication link Z to the device or system to be controlled 10 to affect operation of the device or system. The communication link is preferably a wireless communication link.

The embodiment of the system 800B in FIG. 8B is the same as that in FIG. 8A, except that the signals indicative of the determined response is transmitted directly by the server 500B to the device to be controlled, via a communication link W. In this embodiment, it is preferably that no direct connection is required between the device 200, 300 and the device to be controller 10.

The embodiment of the system 800C in FIG. 8C is the same as that in FIG. 8A, except that the smart phone 700 is omitted. In this embodiment, the device 200, 300 is in direct communication with the server 500C through communication link P. Communication link P is preferably a wireless communication link such as a cellular or Wi-Fi communication link. The server 500C, upon determining the result, transmits the result to the device 200, 300, to allow the device 200, 300 to provide control signal via communication link Q to the system or device to be controlled 10. Communication link Q is preferably a wireless communication link.

The embodiment of the system 800D in FIG. 8D is the same as that in FIG. 8C, except that the signals indicative of the determined response is transmitted directly by the server 500D to the device to be controlled, via a communication link R, preferably wireless. In this embodiment, it is preferably that no direct connection is required between the device 200, 300 and the device to be controller 10.

The server 500, 500A-500D, charger 900, ring accessory 1000, and external electronic device 700 in FIGS. 5-8D may be implemented using one or more of the following example information handling system. The information handling system may have different configurations, and it generally comprises suitable components necessary to receive, store and execute appropriate computer instructions or codes. The main components of the information handling system are a processing unit and a memory unit. The processing unit is a processor such as a CPU, an MCU, etc. The memory unit may include a volatile memory unit (such as RAM), a non-volatile unit (such as ROM, EPROM, EEPROM and flash memory) or both. Optionally, the information handling system further includes one or more input devices such as a keyboard, a mouse, a stylus, a microphone, a tactile input device (e.g., touch sensitive screen) and a video input device (e.g., camera). The information handling system may further include one or more output devices such as one or more displays, speakers, disk drives, and printers. The displays may be a liquid crystal display, a light emitting display or any other suitable display that may or may not be touch sensitive. The information handling system may further include one or more disk drives which may encompass solid state drives, hard disk drives, optical drives and/or magnetic tape drives. A suitable operating system may be installed in the information handling system, e.g., on the disk drive or in the memory unit of the information handling system. The memory unit and the disk drive may be operated by the processing unit. The information handling system also preferably includes a communication module for establishing one or more communication links (not shown) with one or more other computing devices such as a server, personal computers, terminals, wireless or handheld computing devices. The communication module may be a modem, a Network Interface Card (NIC), an integrated network interface, a radio frequency transceiver, an optical port, an infrared port, a USB connection, or other interfaces. The communication links may be wired or wireless for communicating commands, instructions, information and/or data. Preferably, the processing unit, the memory unit, and optionally the input devices, the output devices, the communication module and the disk drives are connected with each other through a bus, a Peripheral Component Interconnect (PCI) such as PCI Express, a Universal Serial Bus (USB), and/or an optical bus structure. In one embodiment, some of these components may be connected through a network such as the Internet or a cloud computing network. The external electronic device may be a mobile phone, a computer, or a tablet. The server may be a cloud computing server that is preferably implemented by combination of software and hardware.

The wearable gesture recognition device and system in the above embodiments of the invention can be connected with different systems and devices, directly or through the server, for controlling these systems and devices. Example applications including:
(1) Smartphone control

The recognized gesture may be used to control operation of the smart phone. For example, fisting the hand would lock the screen of the phone, trigger the phone to capture an image, etc.

(2) Smart home control

The recognized gesture may be used to control operation of the smart phone. For example, fisting the hand would switch off the lights, straightening two fingers may switch on two lights, three fingers three lights, etc.
(3) Music gesture training

The recognized gesture may be used as part of a musician training program to determine posture or even force applied during various instances to assist, for example, violin training.
(4) Sports gesture training

The recognized gesture may be used as part of a sports training program to determine posture or even force applied during various instances to assist, for example, javelin throw training.
(5) VR/AR gaming

The recognized gesture may be used as part of a gaming system as game control (user input).
(6) Sign language translation

The recognized gesture may be used for real-time sign language translation. For example, real time conversion of sign language to text on computer screen, to assist translation of sign language.
(7) Rapid preliminary medical screening

The recognized gesture may be used for real-time preliminary medical screening of disease associated with body parts on which the device is worn. In one specific example, the device can be used for carpal tunnel syndrome (CTS) screening. CTS is a common medical condition that causes pain, numbness, and tingling in the hand and arm, generally caused by compression of the median nerve at the wrist. Existing clinical diagnosis of CTS uses nerve conduction studies and ultrasound in hospitals, which are relatively complicated and require long wait-time (due to the large demand and the relatively little resource in the hospitals). In one example, the wristband provides a portable imaging modality with the capability to capture cross sectional plane of the wrist at high speed (<1 min). As such the cross sectional area of the median nerve within or near the carpal tunnel can be readily measured for assessment.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the invention are either wholly implemented by computing system or partly implemented by computing systems then any appropriate computing system architecture may be utilized. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. For example, the wearable gesture recognition device may take various form not limited to the one illustrated in FIGS. 2-3B. The wearable gesture recognition device need not be wrist worn but may be worn on any other parts of the body of the user. The signal processing may be performed substantially entirely on the wearable gesture recognition device, partly on the wearable gesture recognition device and partly on the server or external electronic device, or substantially entirely on the server or external electronic device. The device or system to be controlled based on the gesture determined can be any electronic device operable to communicate with the wearable gesture recognition device or the server, directly or indirectly. The wearable gesture recognition device may further include an IMU arranged to determine movement of the wearable gesture recognition device to affect determination of the electrical impedance tomogram. The wearable gesture recognition device may further include one or more biosensors arranged to detect physiological signals of the wearer to affect determination of the electrical impedance tomogram. The one or more biosensors may be any of: a blood oxygen level sensor; a pulse rate sensor; a heart rate sensor; and an EMG detector. The wearable gesture recognition device may further include a GPS module arranged to determine location of the wearable gesture recognition device. The determined location may optionally be used to affect determination of the electrical impedance tomogram. The present embodiments are, therefore, to be considered in all respects as illustrative and not restrictive.

The wearable gesture recognition device in some embodiments of the invention can be implemented as a wearable device for medical screening of disease associated with one or more body parts of a user. In some embodiments, there is provided a wearable device operable to facilitate medical screening of disease by collecting electrical (e.g., conductivity) signals from the user for reconstructing an electrical impedance tomogram, without recognizing gesture. The reconstruction of the electrical impedance tomogram and associated medical screening can be performed on an external device external to the wearable device.

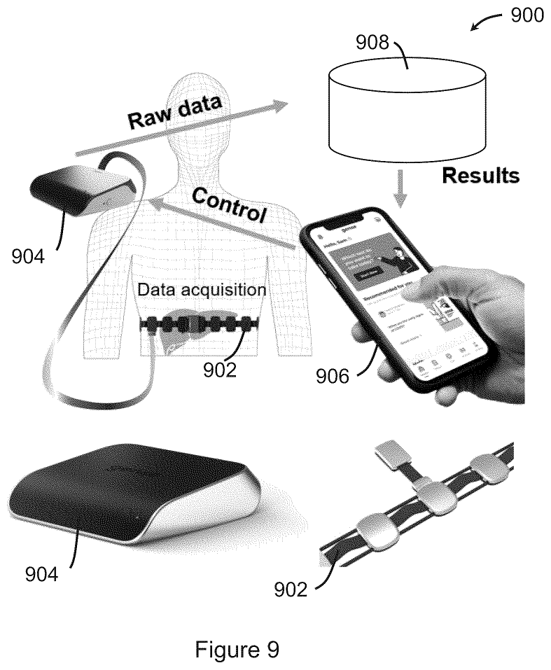

FIG. 9 shows a medical screening system 900 in some embodiments of the invention. In these embodiments, the medical screening system 900 includes a wearable device 902, a portable device 904, a mobile device 906, and a server 908. The wearable device 902 is arranged to be worn on a subject (i.e., user), e.g., around the chest, abdomen, etc., to provide electrical signals to and receive responsive electrical (e.g., conductivity) signals from the subject, hence to facilitate performing of electrical impedance tomography on the subject. The portable device 904 is arranged to be electrically connected with the wearable device 902, for controlling the electrical signals arranged to be provided to the subject via the wearable device 902, and for processing the responsive electrical (e.g., conductivity) signals. The mobile device 906 is arranged to be in communication with the portable device 904 to control operation of the portable device 904. The mobile device 906 may be installed with an application or may be used to access a web application dedicated for controlling the portable device 904. In some examples, the mobile device 906 is a mobile phone. In some examples, the mobile device 906 may be a tablet computer, notebook computer, etc. The server 908 is arranged to be in communication with the portable device 904, for processing signals received from the portable device 904 for determining an electrical impedance tomography data (e.g., electrical impedance tomogram) associated with the subject and for performing medical screening based on the determined electrical impedance tomography data. To this end, the server 908 may use various processing modules to process the electrical impedance tomography data hence to perform medical screening. The server 908 is further arranged to be in communication with the mobile device 906 for providing the processing result (e.g., medical screening result) to the mobile device 906. The mobile device 906 may be installed with an application or may be used to access a web application dedicated for displaying the result provided by the server 908. In some examples, the server 908 is a cloud computing server.

While not illustrated, in some embodiments, the mobile device 906 and the server 908 may be implemented using the same information processing system, e.g., the same computer or computers.

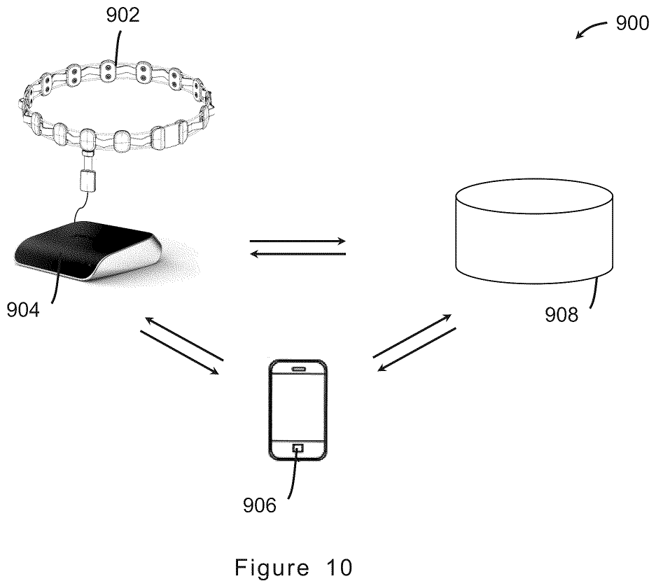

FIG. 10 shows the connections and communication links among various devices of the medical screening system 900 in some embodiments.

As shown in FIG. 10, in these embodiments, the portable device 904 and the wearable device 902 are connected via a wired connection. In some examples, the wired connection between the portable device 904 and the wearable device 902 may be provided at least by HDMI cable(s). In some examples, the wired connection may be provided at least by USB cable(s). Other wired connection is also possible. Data and/or power may be communicated between the portable device 904 and the wearable device 902 via the wired connection.

As shown in FIG. 10, in these embodiments, the portable device 904 is in communication with the mobile device 906 via wired and/or wireless connection. In some examples, the wired and/or wireless connection between the portable device 904 and the mobile device 906 may include at least one of a Bluetooth® connection, a NFC connection, a Wi-Fi connection, a ZigBee connection, a radio frequency (RF) connection, a cellular (2G, 3G, 4G, 5G, 6G or above) connection, etc. Other wired and/or wireless connection are also possible. Data may be communicated between the portable device 904 and the mobile device 906 via the wired and/or wireless connection. For example, control signals may be communicated from the mobile device 906 to the portable device 904.

As shown in FIG. 10, in these embodiments, the portable device 904 is in communication with the server 908 via wired and/or wireless connection. In some examples, the wired and/or wireless connection between the portable device 904 and the server 908 may include at least one of a Bluetooth® connection, a NFC connection, a Wi-Fi connection, a ZigBee connection, a radio frequency (RF) connection, a cellular (2G, 3G, 4G, 5G, 6G or above) connection, etc. Other wired and/or wireless connection are also possible. Data may be communicated between the portable device 904 and the server 908 via the wired and/or wireless connection. For example, signals obtained from the subject via the wearable device 902 may be communicated from the portable device 904 to the server 908 for processing and analysis. For example, signals obtained from the subject via the wearable device 902 may be communicated from the portable device 904 to the server 908 for reconstructing an electric impedance tomogram or for obtaining electric impedance tomography data, hence for performing medical screening (based on the electric impedance tomogram/electric impedance tomography data) based on various processing models.

As shown in FIG. 10, in these embodiments, the mobile device 906 is in communication with the server 908 via wired and/or wireless connection. In some examples, the wired and/or wireless connection between the mobile device 906 and the server 908 may include at least one of a Bluetooth® connection, a NFC connection, a Wi-Fi connection, a ZigBee connection, a radio frequency (RF) connection, a cellular (2G, 3G, 4G, 5G, 6G or above) connection, etc. Other wired and/or wireless connection are also possible. Data may be communicated between the mobile device 906 and the server 908 via the wired and/or wireless connection. For example, processing results obtained at the server 908 may be communicated to the mobile device 906 for further processing and/or for display.

In some embodiments, the mobile device 906 and the server 908 may be implemented using (e.g., replaced with) a single information processing system, e.g., the same computer or computers. In such case, the information processing system may communicate with the portable device 904 much like the mobile device 906 and the server 908.

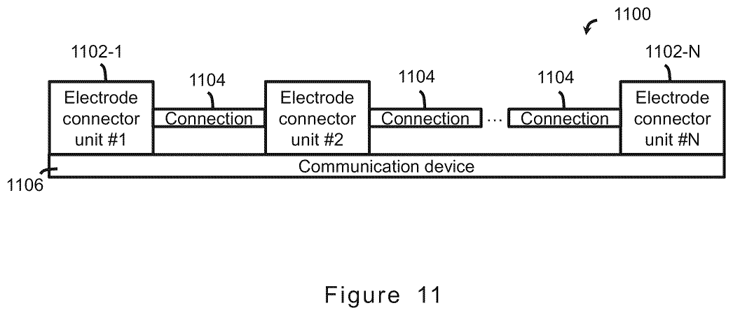

FIG. 11 illustrates a wearable device 1100 for facilitating performing of electrical impedance tomography in some embodiments of the invention. The wearable device 1100 can be considered as examples of the wearable device 902.

In these embodiments, the wearable device 1100 has N (N≥4) electrode connector units 1102-1 to 1102-N each arranged for electrically connecting with a respective electrode. The electrodes may be removably connectable with the electrode connector units 1102-1 to 1102-N. The electrodes may be disposable, reusable, or for single-use only. The electrodes may include gel electrodes and/or dry electrodes, e.g., in the form of electrode pads. The wearable device 1100 also includes a connection arrangement 1104 that connects the electrode connector units 1102-1 to 1102-N. The connection arrangement may include a mechanical connection arrangement for mechanically connecting the electrode connector units 1102-1 to 1102-N and an electrical connection arrangement for electrically connecting the electrode connector units 1102-1 to 1102-N. The wearable device 1100 also includes a communication device 1106 that is operably connected with the electrode connector units 1102-1 to 1102-N. The communication device 1106 is arranged to enable communication between the electrode connector units 1102-1 to 1102-N and a portable device (such as portable device 904) arranged to facilitate performing of electrical impedance tomography. The communication device 1106 may enable data and/or power communication between the electrode connector units 1102-1 to 1102-N and the portable device. The wearable device 1100 may be in the form of a belt or a band arranged to be worn on a body of a subject (e.g., chest, abdomen, waist, head, wrist, etc., of the subject). The belt or band, when worn, may be in the form of a closed loop.

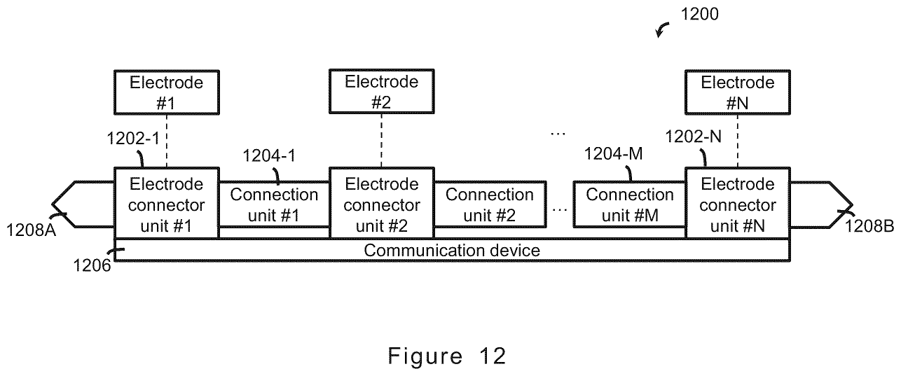

FIG. 12 shows a wearable device 1200 for electrical impedance tomography in some embodiments of the invention. The wearable device 1200 can be considered as example implementations of the wearable device 1100.

The wearable device 1200 includes N (N≥4) electrode connector units 1202-1 to 1202-N each arranged for electrically connecting with a respective electrode. The electrodes may be removably connectable with the electrode connector units 1202-1 to 1202-N. The electrodes may include gel electrodes and/or dry electrodes, e.g., in the form of electrode pads. The electrode connector units 1202-1 to 1202-N may each include: a housing, one or more electrode connectors arranged in or on the housing for electrically connecting with an electrode (e.g., corresponding one or more connectors of the electrode), and a circuit arranged in the housing and electrically connected with the electrode connector. In some implementations, the housing includes at least two housing parts (components) removably connected with each other, e.g., via complementary engagement means such as fastener arrangement, press fit arrangement, snap fit arrangement, magnetic mechanism, etc. The at least two housing parts may include a base and a cover removably connectable to the base. The cover or the base may include one or more openings. Each of the one or more electrode connectors may be arranged in a respective opening or extends at least partly through a respective opening, such that the electrode connector can be accessed for connection of electrode. The housing may be made of plastic material(s). In some implementations, the electrode connector includes a snap connector (e.g., one of a male connector and a female connector) for connecting with a corresponding snap connector (e.g., another one of a male connector and a female connector) of the electrode. The electrode connector may be made of electrically conductive material(s), e.g., metal, alloy such as stainless steel, etc. In some implementations, the circuit is arranged in or on a circuit board arrangement, e.g., a printed circuit board assembly (PCBA), arranged in the housing. The printed circuit board assembly may be removably arranged (e.g., mounted) in the housing. The printed circuit board assembly may be mounted with the electrode connector(s), which the circuit electrically connects with. The printed circuit board assembly may also include one or more circuit connection interfaces for enabling electrical connection with the circuit.

The wearable device 1200 also includes a connection arrangement that connects the electrode connector units 1202-1 to 1202-N. The connection arrangement may include a mechanical connection arrangement for mechanically connecting the housings of the electrode connector units 1202-1 to 1202-N and an electrical connection arrangement for electrically connecting the circuits of the electrode connector units 1202-1 to 1202-N. As shown in FIG. 12, the connection arrangement includes M (M≥3) connection unit(s) 1204-1 to 1204-M each arranged between respective adjacent electrode connector units 1202-1 to 1202-N. In some implementations, M=N−1. The connection unit(s) may each include one or more first connectors for connecting the corresponding adjacent electrode connector units. In some implementations, the first connector includes a flexible and/or elastic body for mechanically connecting the housings of the corresponding adjacent electrode connector units and a flexible and/or elastic circuit coupled to (e.g., overmolded with) the flexible and/or elastic body for electrically connecting the circuits of the corresponding adjacent electrode connector units. The first connector may be removably connected with one or both of the corresponding adjacent electrode connector units. The first connector may define a first circuit connector connecting with a circuit connection interface of one of the corresponding adjacent electrode connector units and a second circuit connector for connecting with a circuit connection interface of another one of the corresponding adjacent electrode connector units. The first and second circuit connectors may be arranged at two ends of the first connector (e.g., with respect to a length of the first connector). The flexible and/or elastic body may include a curved or undulating portion that can become less curved or less undulated (e.g., straightened) when the corresponding adjacent electrode connector units move relatively away from each other. The connection unit(s) may each further include, in addition to the first connector, one or more second connectors for mechanically connecting the corresponding adjacent electrode connector units. The second connector may lack electrical arrangement or components. In some implementations, the second connector includes a flexible and/or elastic body for mechanically connecting the housings of corresponding adjacent electrode connector units. The second connector may be less flexible and/or more elastic than the first connector. The second connector may be removably connected, non-removably connected, or integral with one or both of the corresponding adjacent electrode connector units. The second connectors of two or more connection units may be connected or integrally formed (e.g., belong to different sections of a longer connector). In some implementations, each connection unit 1204-1 to 1204-M respectively includes one first connector and two second connectors.

The wearable device 1200 also includes a communication device 1206 that is operably connected with the electrode connector units 1202-1 to 1202-N. The communication device 1206 is arranged to enable communication between the electrode connector units 1202-1 to 1202-N and a portable device (e.g., portable device 904) arranged to facilitate performing of electrical impedance tomography. The communication device 1206 may enable data and/or power communication between the electrode connector units 1202-1 to 1202-N and the portable device. In some implementations, the communication device 1206 includes a wired communication device for enabling wired communication between the electrode connector units 1202-1 to 1202-N and the portable device. The wired communication device may include a connector (e.g., plug, socket, port, receptacle, etc.) for connecting with a corresponding connector of the portable device. As an example, the wired communication device may include an HDMI communication device and the connector may include an HDMI connector. As another example, the wired communication device may include a USB communication device and the connector may include a USB connector. In some implementations, the communication device 1206 additionally or alternatively includes a wireless communication device for enabling wireless communication between the electrode connector units 1202-1 to 1202-N and the portable device. For example the communication device 1206 may include a modem, a Network Interface Card (NIC), an integrated network interface, a NFC transceiver, a ZigBee transceiver, a Wi-Fi transceiver, a Bluetooth® transceiver, a radio frequency transceiver, a cellular (2G, 3G, 4G, 5G, 6G or above, or the like) transceiver, or etc. The transceiver may be implemented by integrated transmitter(s) and receiver(s), separate transmitter(s) and receiver(s), or etc.

The wearable device 1200 also includes a coupler arrangement arranged to facilitate wearing of the wearable device 1200 by the subject. The coupler arrangement may include two couplers 1208A, 1208B each arranged at or near a respective end of the wearable device 1200. The couplers 1208A, 1208B may be removably coupled with each other. In some implementations, the couplers 1208A, 1208B are buckle members that are releasably engageable. The coupler 1208A is coupled to a housing of the electrode connector unit 1202-1 at one end of the wearable device 1200 and the coupler 1208B is coupled to a housing of the electrode connector unit 1202-N at another end of the wearable device 1200.

The wearable device 1200 may be in the form of a belt or a band arranged to be worn on a body of a subject (e.g., chest, abdomen, waist, head, wrist, etc., of the subject). This can facilitated by the coupler arrangement or other arrangement(s). The wearable device 1200 can be made liquid (e.g., water) resistant.

In some implementations, the circuits of the electrode connector units 1202-1 to 1202-N and the electrical connection arrangement of the connection arrangement are arranged in the same circuit, which may be arranged on or in a single flexible circuit. In some implementations, the housings of the electrode connector units 1202-1 to 1202-N and the mechanical connection arrangement of the connection arrangement are provided by multiple (e.g., two) housing members each of which is integrally formed. In this way, each of the housing members may provide a portion of each of the housing of the electrode connector units 1202-1 to 1202-N. In some other implementations, electrodes may be integrated with (e.g., non-removably connected with) the electronic connector units 1202-1 to 1202-N.

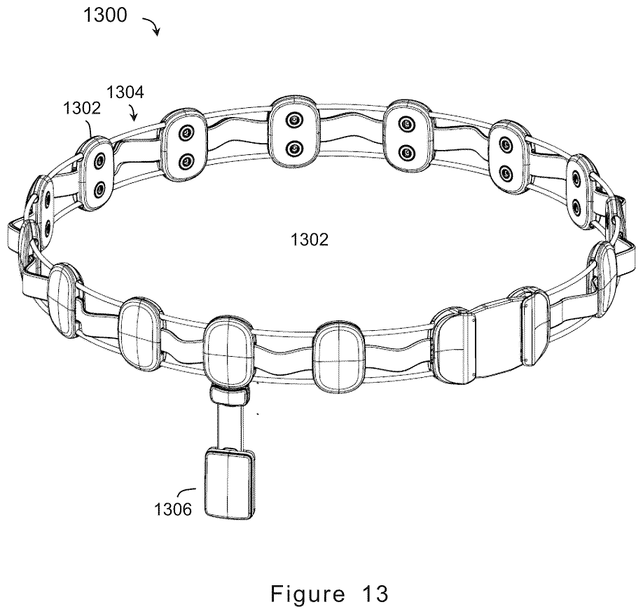

FIG. 13 shows a wearable device 1300 for electrical impedance tomography in one embodiment of the invention. The wearable device 1300 is a specific implementation of the wearable device 1200, thus it includes the features of the wearable device 1200. The wearable device 1300 generally includes: multiple electrode connector units (only one is annotated using 1302 in FIG. 13) each arranged for electrically connecting with a respective electrode, a connection arrangement with multiple connectors (only one set is annotated using 1304 in FIG. 13) connecting the electrode connector units, and a communication device 1306 operably connected with the electrode connector units and arranged to enable communication between the electrode connector units and a portable device arranged to facilitate performing of electrical impedance tomography. For brevity, other features of the wearable device 1300 have been described with reference to wearable device 1200, hence will not be repeated here.

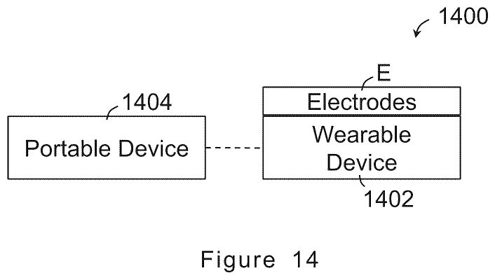

FIG. 14 shows a portable system 1400 for facilitating performing of electrical impedance tomography in some embodiments of the invention. The portable system 1400 generally includes a wearable device 1402 with electrodes E arranged to be worn by a user to provide electrical signals to the user and to obtain responsive electrical signals from the user, and a portable device 1404 for facilitating performing of electrical impedance tomography. In some examples, the electrodes E may be removably connected with the wearable device 1402. In some examples, the electrodes E may be non-removably connected with the wearable device 1402. The wearable device 1402 may be the wearable device 902, 1100, 1200, 1300. The portable device 1404 may be the portable device 904. The portable device 1404 is arranged to generate and transmit electrical signals to the user via the wearable device 1402 and the electrodes E, and to receive and process responsive electrical signals from the user via the electrodes E and the wearable device 1402.

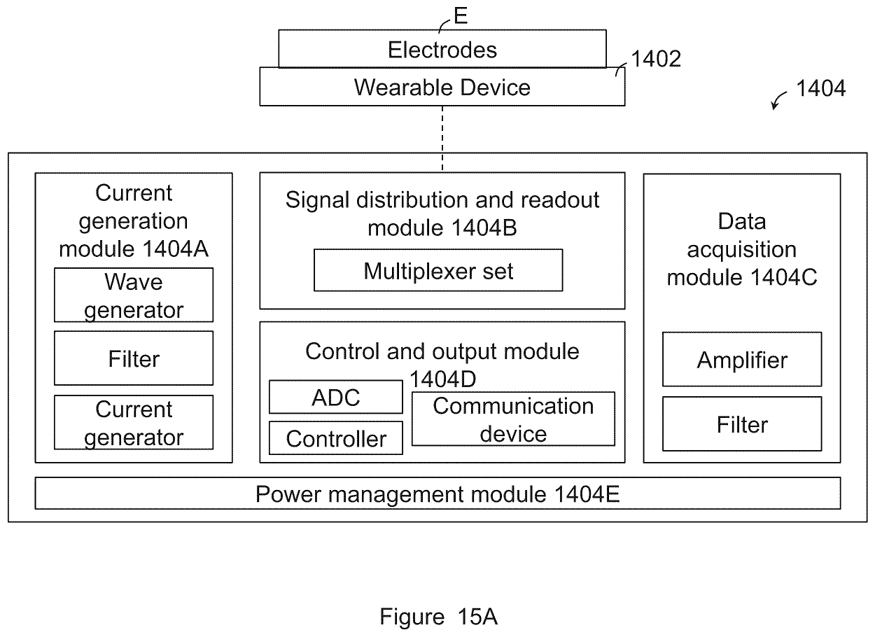

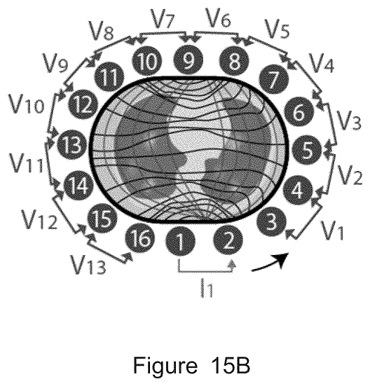

FIG. 15A is the portable device 1404 of the portable system 1400 in some embodiments of the invention. The portable device 1404 generally includes, at least, a current generation module 1404A, a signal distribution and readout module 1404B, a data acquisition module 1404C, a control and output module 1404D, and a power management module 1404E.

The current generation module 1404A is arranged to generate electric current signals for providing to the subject. In these embodiments, the current generation module 1404A may include a waveform generator arranged to generate wave signals, an optional filter arranged to reduce harmonic distortion and/or electromagnetic interference of the generated wave signals, and a current generator arranged to generate electric current signals (wave-modulated) based on the filtered wave signals. The generation of wave signals by the waveform generator may be controlled by the controller in the control and output module 1404D. The generated electric current signals may be sinusoidal current signals.

The signal distribution and readout module 1404B is arranged to receive the generated electric current signals from the current generation module 1404A and to provide the generated electric current signals (e.g., sinusoidal current signals) to the subject via the wearable device 1402 and the electrodes E. The signal distribution and readout module 1404B is further arranged to receive responsive electrical signals (e.g., voltage/electric potential signals) from the subject via the electrodes E and the wearable device 1402. In these embodiments, the signal distribution and readout module 1404B includes a multiplexer set, with one or more N:1 multiplexers, where N is an integer corresponding to the total number of electrodes used. For example, N may equal to 16 or 32. In some embodiments, at least one of the N:1 multiplexers is used for providing the generated electric current signals to the subject. In some embodiments, at least one of the N:1 multiplexers is used for readout. The signal transmission and readout operation of the signal distribution and readout module 1404B is arranged to be controlled by the controller in the control and output module 1404D. In some embodiments, the signal transmission and readout operation is based on adjacent pattern measurement protocol (adjacent stimulation and measurement patterns), wherein sinusoidal current is applied between a pair of adjacent electrodes and boundary potential is measured between all other pairs of adjacent electrodes and the process is repeated for all pairs of adjacent electrodes (i.e., the total number of times sinusoidal current application equals to the total number of electrodes), as schematically illustrated in FIG. 15B.

The data acquisition module 1404C is arranged to obtain, determine, and amplify the potential differences obtained from the electrodes E in response to the providing electric current signals to the subject. In these embodiments, the data acquisition module 1404C includes a data acquisition amplifier, e.g., a multi-stage data acquisition amplifier, and a filter, e.g., a bandpass filter.

The control and output module 1404D is arranged to control the providing of electrical current signals to the user and to control the receiving of electrical signals from the user. The control and output module 1404D is further arranged to process the processed potential differences signals received from the data acquisition module 1404C and to transmit the processed signals to a server for further processing and analysis (e.g., for obtaining electrical impedance tomography data and for medical screening). In these embodiments, the control and output module 1404D includes an analog-to-digital converter (ADC) for digitizing the processed potential differences signals received from the data acquisition module 1404C. In these embodiments, the control and output module 1404D also includes a controller arranged to control the generation of wave signals by the waveform generator in the current generation module 1404A, to control the signal transmission and readout operation of the signal distribution and readout module 1404B, and to process the digitized signal from the analog-to-digital converter. In these embodiments, the control and output module 1404D also includes a communication device for communicating the processed data to the server. The communication device may include a wired and/or wireless communication device. The communication device may include one or more of: a modem, a Network Interface Card (NIC), an integrated network interface, a NFC transceiver, a ZigBee transceiver, a Wi-Fi transceiver, a Bluetooth® transceiver, a radio frequency transceiver, a cellular (2G, 3G, 4G, 5G, above 5G, or the like) transceiver, an optical port, an infrared port, a USB connection, or other wired or wireless communication interfaces.

The power management module 1404E is arranged to manage power provided to the current generation module 1404A, the signal distribution and readout module 1404B, the data acquisition module 1404C, the control and output module 1404D, and optionally one or other modules of the portable device 1404 not illustrated, for operating the portable device 1404. In these embodiments, the power management module 1404E includes a power circuit arranged to be electrically connected with a power source (e.g., battery or AC mains).

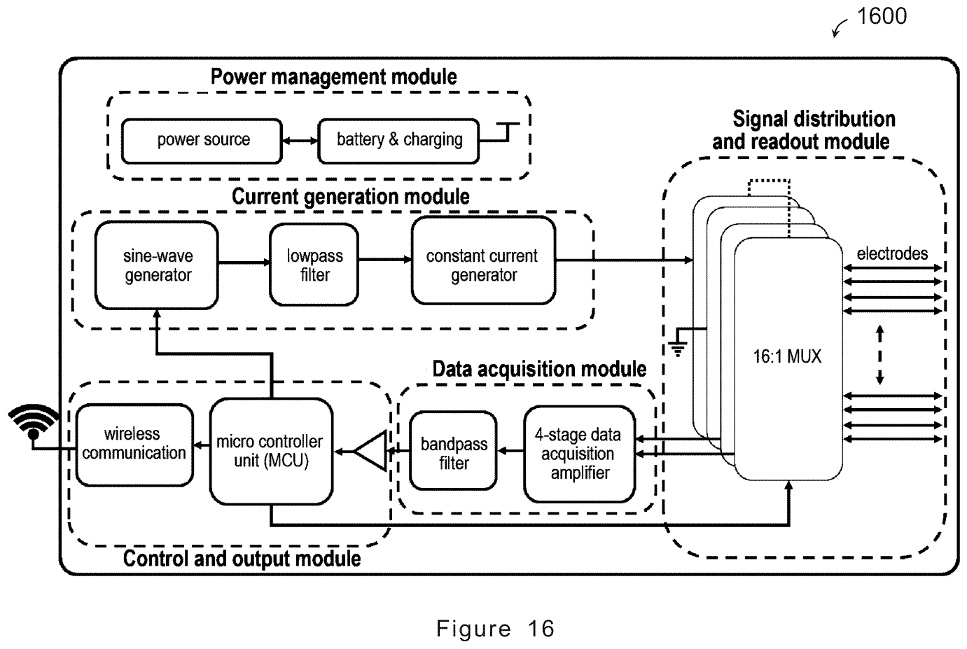

FIG. 16 shows a portable device 1600 for facilitating performing of electrical impedance tomography in some embodiments of the invention. The portable device 1600 can be considered as a specific implementation of the portable device 1404 of FIG. 15A.

Referring to FIG. 16, the portable device 1600 generally includes, at least, a power management module for constant power supply, a current generation module for alternating current generation, a signal distribution and readout module for current injection and voltage/potential readout, a data acquisition module for potential difference measurement, amplification, and acquisition, and a control and output module for module coordination, data processing, and cloud-based server communication.

In the portable device 1600, the power management module that provides power supply to all other modules through the power socket or battery (e.g., Li-ion battery).

In the portable device 1600, the current generation module mainly includes a sine wave generator and a constant current generator successively to generate an alternating current (e.g., of 1 mApp) and a voltage (e.g., amplitude of 1 Vpp). The current generation module may also include a low-pass filter to suppress total harmonic distortion and ambient electromagnetic interference (e.g., power line noise).

In the portable device 1600, the signal distribution and readout module is arranged to introduce the generated current to the subject via 16-electrodes mounted to the wearable device 1402 using a set of CMOS multiplexers (MUXs). In one example, four MUXs are used, in which two MUXs are employed for current injection and the other two for voltage/potential readout. The MUXs may be configured into the adjacent-scan pattern through the microcontroller unit (MCU) in the control and output module.

In the portable device 1600, the data acquisition module is the analog front-end (AFE) that acquires, measures and amplifies the potential differences from the electrodes. In one example, the AFE comprises a four-stage wide input differential amplifier with high common-mode rejection ratio (CMRR), and a bandpass filter.

In the portable device 1600, the control and output module includes an analog-to-digital converter (ADC), a microcontroller unit (MCU) and a wireless communication chip. The potential differences obtained from the data acquisition module are digitized by the ADC (e.g., 12-bit ADC), processed in the MCU, and transferred to the cloud-based server for further processing and analysis (e.g., for obtaining electrical impedance tomography data and for medical screening).

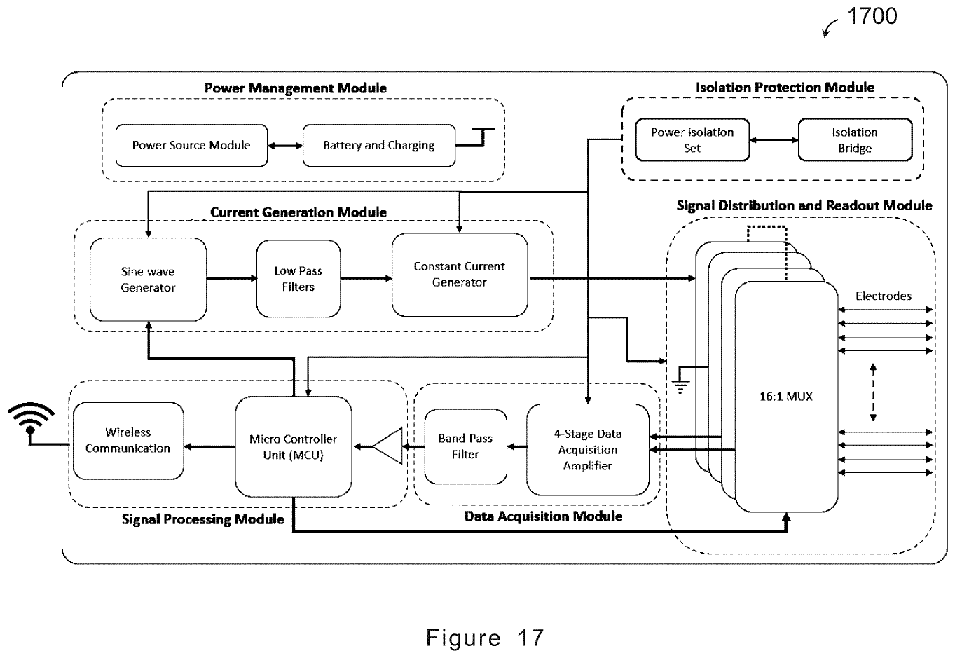

FIG. 17 shows a portable device 1700 for facilitating performing of electrical impedance tomography in some embodiments of the invention. The portable device 1700 can be considered as another specific implementation of the portable device 1404 of FIG. 15A.

Like the portable device 1600, the portable device 1700 also generally includes, at least, a power management module for constant power supply, a current generation module for alternating current generation, a signal distribution and readout module for current injection and voltage/potential readout, a data acquisition module for potential difference measurement, amplification, and acquisition, and a control and output module for module coordination, data processing, and cloud-based server communication. These various modules in portable device 1700 can be constructed generally the same as those in portable device 1600 so for brevity they are not described in detail here.

Unlike the portable device 1600, the portable device 1700 includes an additional isolation protection module for improving safety and effectiveness of the portable device 1700. The isolation protection module generally includes an isolation bridge operably coupled with a power isolation set/circuit. In some embodiments, the isolation bridge is implemented with a clipping distance of about 4 mm. In some embodiments, the power isolation set/circuit includes an isolation transformer, and various active and passive circuit components. The isolation protection module is electrically connected with each of the current generation module (e.g., its wave generator and current generator), the signal distribution and readout module, the data acquisition module (e.g., its data acquisition amplifier), and the control and output module (e.g., its MCU).

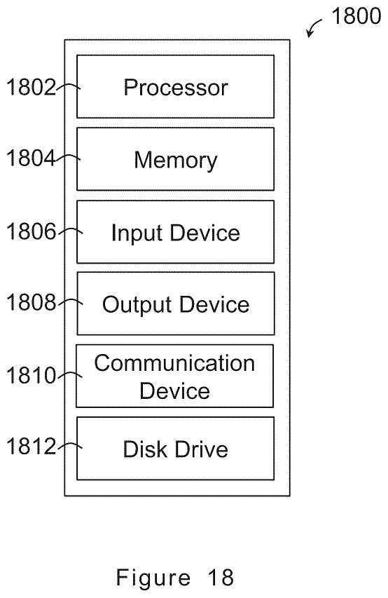

FIG. 18 shows an information handling system 1800 in some embodiments of the invention. The information handling system 1800 may be the mobile device 906, the server 908, an information processing system the replaces (assumes the role of) both the mobile device 906 and the server 908, etc.

The information handling system 1800 generally comprises suitable components necessary to receive, store, and execute appropriate computer instructions, commands, and/or codes. The main components of the information handling system 1800 are a processor 1802 and a memory (storage) 1804. The processor 1802 may include one or more of: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), or any other digital or analog circuitry/circuitries configured to interpret and/or to execute program instructions and/or to process signals and/or information and/or data. The memory 1804 may include one or more volatile memory (such as RAM, DRAM, SRAM, etc.), one or more non-volatile memory (such as ROM, PROM, EPROM, EEPROM, FRAM, MRAM, FLASH, SSD, NAND, NVDIMM, etc.), or any of their combinations. Appropriate computer instructions, commands, codes, information and/or data may be stored in the memory 1804. Computer instructions for executing or facilitating executing the method embodiments of the invention may be stored in the memory 1804. For example, the information handling system 1800 may store in the memory 1804 an application for controlling operation of the portable device 904, 1404, 1600, 1700. For example, the information handling system 1800 may store in the memory 1804 various processing algorithms or routines (machine learning based and/or non machine learning based) for processing signals outputted by the portable device 904, 1404, 1600, 1700 for obtaining electrical impedance tomography data (e.g., electrical impedance tomogram) and for performing medical screening based on the obtained electrical impedance tomography data. The processor 1802 and memory (storage) 1804 may be integrated or separated (and operably connected). Optionally, the information handling system 1800 further includes one or more input devices 1806. Example of such input device 1806 include: keyboard, mouse, stylus, image scanner, microphone, tactile/touch input device (e.g., touch sensitive screen), image/video input device (e.g., camera), etc. Optionally, the information handling system 1800 further includes one or more output devices 1808. Example of such output device 1808 include: display (e.g., monitor, screen, projector, etc.), speaker, headphone, earphone, printer, additive manufacturing machine (e.g., 3D printer), etc. The display may include a LCD display, a LED/OLED display, or other suitable display, which may or may not be touch sensitive. The information handling system 1800 may further include one or more disk drives 1812 which may include one or more of: solid state drive, hard disk drive, optical drive, flash drive, magnetic tape drive, etc. A suitable operating system may be installed in the information handling system 1800, e.g., on the disk drive 1812 or in the memory 1804. The memory 1804 and the disk drive 1812 may be operated by the processor 1802. Optionally, the information handling system 1800 also includes a communication device 1810 for establishing one or more communication links (not shown) with one or more other computing devices, such as servers, personal computers, terminals, tablets, phones, watches, IoT devices, or other wireless computing devices. The communication device 1810 may include one or more of: a modem, a Network Interface Card (NIC), an integrated network interface, a NFC transceiver, a ZigBee transceiver, a Wi-Fi transceiver, a Bluetooth® transceiver, a radio frequency transceiver, a cellular (2G, 3G, 4G, 5G, above 5G, or the like) transceiver, an optical port, an infrared port, a USB connection, or other wired or wireless communication interfaces. Transceiver may be implemented by one or more devices (integrated transmitter(s) and receiver(s), separate transmitter(s) and receiver(s), etc.). The communication link(s) may be wired or wireless for communicating commands, instructions, information and/or data. In one example, the processor 1802, the memory 1804 (optionally the input device(s) 1806, the output device(s) 1808, the communication device(s) 1810 and the disk drive(s) 1812, if present) are connected with each other, directly or indirectly, through a bus, a Peripheral Component Interconnect (PCI), such as PCI Express, a Universal Serial Bus (USB), an optical bus, or other like bus structure. In one embodiment, at least some of these components may be connected wirelessly, e.g., through a network, such as the Internet or a cloud computing network. A person skilled in the art would appreciate that the information handling system 1800 shown in FIG. 18 is merely an example and that the information handling system 1800 can, in other embodiments, have different configurations (e.g., has additional components, has fewer components, etc.).

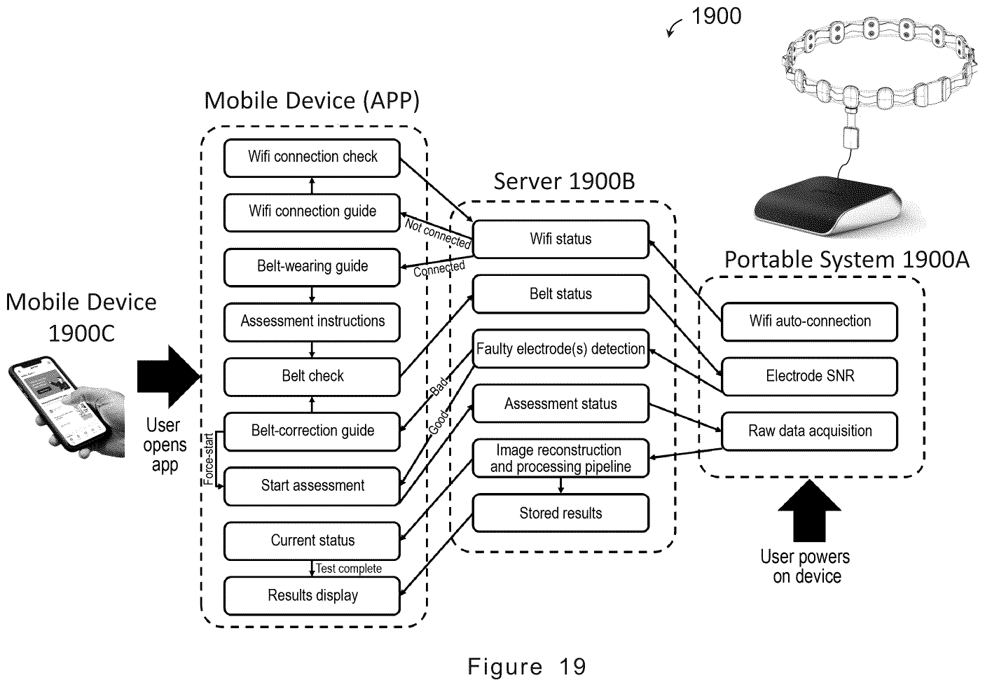

FIG. 19 illustrates operation of a medical screening system 1900 in some embodiments of the invention. The medical screening system 1900 includes a portable system 1900A for facilitating performing of electrical impedance tomography, a server 1900B, and a mobile device 1900C installed with an application. The portable system 1900A may be implemented using the wearable device 902, 1100, 1200, 1300, 1402 and the portable device 904, 1404, 1600, 1700. The server 1900B may be implemented using the information handling system 1800. The mobile device 1900C may be implemented using the information handling system 1800.

The basic operation is as follows. First, the portable device of the portable system 1900A is connected to Wi-Fi network, either automatically or manually. Then, through the application on the mobile device 1900C, and the server 1900B, the user will be guided to wear the wearable device (e.g., belt), and thereafter, electrodes connection quality will be determined and analysed. Once an acceptable signal-to-noise ratio (SNR) (e.g., above a threshold) is achieved, the data (electrical potential) acquisition will be performed via the portable system 1900A. The data acquired by the portable system 1900A is then transferred to the image reconstruction and processing pipeline in the server 1900B for processing, and the processing results (medical screening result) will be transmitted to the mobile device 1900C for display.

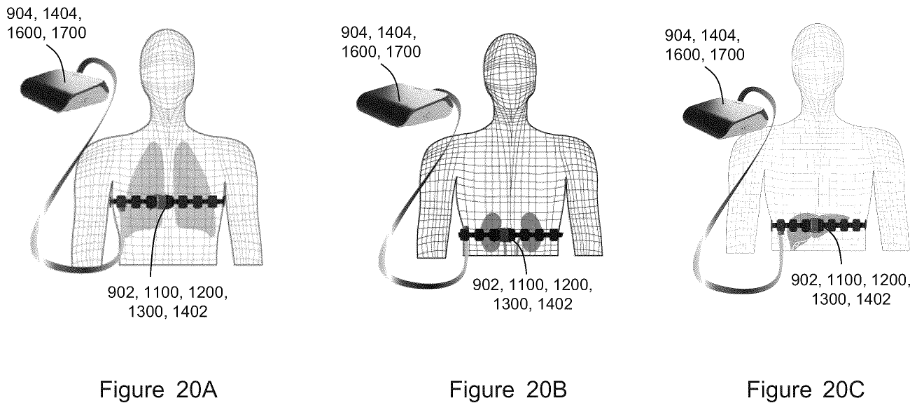

FIGS. 20A to 20C show three example uses of the portable system for facilitating performing of electrical impedance tomography. The portable system may be implemented using the wearable device 902, 1100, 1200, 1300, 1402 and the portable device 904, 1404, 1600, 1700. In FIG. 20A, the portable system is used for performing lung function detection or disease screening. In FIG. 20B, the portable system is used for performing kidney function detection or disease screening. In FIG. 20C, the portable system is used for performing liver function detection or disease screening.

Although not required, one or more embodiments described with reference to the Figures can be implemented as an application programming interface (API) or as a series of libraries for use by a developer or can be included within another software application, such as a terminal or computer operating system or a portable computing device operating system. In one or more embodiments, as program modules include routines, programs, objects, components, and data files assisting in the performance of particular functions, the skilled person will understand that the functionality of the software application may be distributed across a number of routines, objects and/or components to achieve the same functionality desired herein.

It will also be appreciated that where the methods and systems of the invention are either wholly implemented by computing system or partly implemented by computing systems then any appropriate computing system architecture may be utilized. This will include stand-alone computers, network computers, dedicated or non-dedicated hardware devices. Where the terms “computing system” and “computing device” are used, these terms are intended to include (but not limited to) any appropriate arrangement of computer or information processing hardware capable of implementing the function described.

It will be appreciated by a person skilled in the art that variations and/or modifications may be made to the described and/or illustrated embodiments of the invention to provide other embodiments of the invention. The described /or illustrated embodiments of the invention should therefore be considered in all respects as illustrative, not restrictive. Example optional features of some embodiments of the invention are provided in the summary and the description. Some embodiments of the invention may include one or more of these optional features (some of which are not specifically illustrated in the drawings). Some embodiments of the invention may lack one or more of these optional features (some of which are not specifically illustrated in the drawings). The medical screening system can be used for performing medical screening of different diseases or health conditions, e.g., diseases or health conditions associated with heart, lung, liver, kidney, etc.

1. A portable device of a medical screening system, comprising:
a current generation module arranged to generate electric current signals for providing to a subject;
a signal distribution and readout module arranged to
receive and provide the generated electric current signals to the subject via a wearable device with electrodes, and
receive responsive electric signals from the subject via the wearable device with electrodes;
a data acquisition module arranged to process responsive electric potential signals received from the subject to determine potential difference signals; and
a control and output module arranged to process the potential difference signals and transmit the processed signals to a server for determining electrical impedance tomography data and medical screening result.

2. The portable device of claim 1, wherein the portable device further comprises:
an isolation protection module electrically connected with the current generation module, the signal distribution and readout module, the data acquisition module, and the control and output module.

3. The portable device of claim 2, wherein the isolation protection module comprises:
an isolation bridge; and
a power isolation circuit operably coupled with the isolation bridge.

4. The portable device of claim 2, wherein the current generation module comprises a waveform generator and a current generator operably connected with the waveform generator.

5. The portable device of claim 4, wherein the current generation module further comprises a filter operably coupled between the waveform generator and the current generator, wherein the filter is arranged to reduce harmonic distortion and/or electromagnetic interference of wave signals generated by the waveform generator.

6. The portable device of claim 4, wherein the waveform generator comprises a sinusoidal waveform generator.

7. The portable device of claim 2, wherein the signal distribution and readout module comprises a plurality of N:1 multiplexers, wherein N is an integer.

8. The portable device of claim 7, wherein N is 16 or 32.

9. The portable device of claim 7, wherein at least one of the plurality of N:1 multiplexers is for signal distribution and at least one of the plurality of N:1 multiplexers is for readout.

10. The portable device of claim 2, wherein the signal distribution and readout module is arranged to operate based on an adjacent pattern measurement protocol.

11. The portable device of claim 2, wherein the data acquisition module comprises a data acquisition amplifier and a filter.

12. The portable device of claim 11,
wherein the data acquisition amplifier comprises a multi-stage data acquisition amplifier; and
wherein the filter comprises a bandpass filter.

13. The portable device of claim 2, wherein the control and output module is further arranged to control operation of the current generation module and the signal distribution and readout module.

14. The portable device of claim 2, wherein the control and output module comprises:
an analog-to-digital converter for digitizing the potential difference signals received from the data acquisition module;
a controller arranged to process the digitized signals; and
a communication device operably connected with the controller for communicating the processed signals to the server.

15. The portable device of claim 14, wherein the controller is further arranged to control operation of the current generation module and the signal distribution and readout module.

16. The portable device of claim 2, wherein the portable device further comprises:
a power management module arranged to manage power provided to the current generation module, the signal distribution and readout module, the data acquisition module, and the control and output module.

17. The portable device of claim 16, wherein the power management module comprises a power circuit arranged to be electrically connected with a power source.

18. A portable system of a medical screening system, comprising:
the portable device of claims 1; and
a wearable device with electrodes arranged to be worn on a body of a user, the wearable device being electrically connectable to the portable device.

19. The portable system of claim 18, where the electrodes are removably connected with the wearable device.

20. A medical screening system comprising:
the portable system of claim 18, and
one or more processors for processing processed signals received from the portable system for determining electrical impedance tomography data and medical screening result.
